(19) United States
(12) Patent Application Publication
(10) Pub. No.: US 2013/0045195 A1
(43) Pub. Date: Feb. 21, 2013

(54) Proteases for Degrading Gluten

(71) Applicant: Pawan Kumar, Belmont, CA (US)

(72) Inventor: Pawan Kumar, Belmont, CA (US)

(73) Assignee: ALVINE PHARMACEUTICALS, INC., San Carlos, CA (US)

(21) Appl. No.: 13/636,980

(22) PCT Filed: Mar. 30, 2011
(86) PCT No.: PCT/US11/30472

§ 371 Date: Nov. 1, 2012

Related U.S. Application Data
(60) Provisional application No. 61/319,178, filed on Mar. 30, 2010.

(51) Int. Cl. A61K 38/48; A23L 1/105; A61P 1/14; C12N 9/64; C12N 15/63

(52) U.S. Cl. 424/94.63; 435/226; 435/320.1; 426/63; 426/18

(57) ABSTRACT

Gluten-degrading proteases derived from insects, including flour beetles, are isolated, and the purified, and recombinant forms can be used to make gluten-containing food safe for patients suffering from gluten intolerance.

### BACKGROUND OF THE INVENTION

1. Field of the Invention

The present invention provides isolated, purified, and recombinant forms of gluten-degrading proteases and methods for their use in degrading gluten in food. The invention therefore relates to the fields of biology, food preparation, medicine, and molecular biology.

2. Description of Related Disclosures

Celiac disease, also known as celiac sprue, and dermatitis herpetiformis (“DH”) are autoimmune diseases (and may be different manifestations of the same disease), and gluten sensitivity is a condition (collectively, celiac disease, DH, and gluten sensitivity are referred to herein as “gluten intolerance”) triggered by dietary gluten, a storage protein found in wheat and other cereals. Patients diagnosed with gluten intolerance are advised or choose on their own to refrain from consuming gluten in any amount. Because gluten is a common protein in food, however, patients find it very difficult to avoid gluten and frequently experience relapse due to inadvertent disclosure.

U.S. Pat. No. 7,303,871 describes therapies for gluten intolerance that involve pretreatment of gluten-containing food with a protease as well as the use of orally administered proteases to degrade gluten contemporaneously with its ingestion. U.S. Pat. No. 7,320,788 describes admixtures of proteases useful in these therapies, including an admixture of a prolyl endopeptidase (PEP), such as Sphingomonas capsulata PEP, and a glutamine endoprotease, such as EPB2 from barley. One such admixture formulated for oral administration and composed of recombinant forms of the barley EPB2 and the S. capsulata PEP (termed, respectively, ALV001 and ALV002; see PCT Pub. Nos. 2008/1115411 and 2008/115428) is currently in clinical trials. Each of the aforementioned patents and patent publications is specifically incorporated herein by reference.

To be effective upon oral administration, a protease must be active or, if in a zymogen form, activate and remain active long enough to degrade any gluten present into non-immunogenic fragments. The immunogenic peptides can be relatively small (˜10 amino acids) and are contained, often in multiple copies, in very large proteins. The conditions in the gastrointestinal tract are harsh, and any exogenously added protease is typically degraded, and so rendered inactive, quickly. Accordingly, there remains a need in the art for proteases useful in the treatment of gluten intolerance. The present invention meets that need.

### SUMMARY OF THE INVENTION

In a first aspect, the present invention provides gluten-degrading, proline-specific proteases from eukaryotic cells, including but not limited to insect cells, including but not limited to proteases from insects that derive protein from dried grain products. Such insects include, without limitation, flour beetles, e.g. members of the darkling beetle genera Tribolium or Tenebrio, which are pests of cereal silos. Species of interest for obtaining gluten-degrading proteases useful in the methods and compositions of the invention include Tribolium castaneum (red flour beetle), Tenebrio molitor (yellow meal worm and other organisms that consume proteins from dried grain products, particularly gluten-containing products, during their development, in isolated, purified, and recombinant form. Proteases of the invention are also provided, in some embodiments, in PEGylated form; see PCT Pub. No. 2007/047303, incorporated herein by reference.

In a second aspect, the present invention provides recombinant expression vectors for the proteases of the invention and methods for using such vectors to produce the encoded proteases.

In a third aspect, the present invention provides methods for degrading gluten in food, comprising contacting gluten-containing food with a protease of the invention in an isolated, purified, or recombinant form. Such methods also include the use of the proteases in combinations, including combinations of two or more proteases derived from insect cells. In other embodiments the insect-derived protease may be combined with a non-insect protease, e.g. Hordeum vulgarum endopeptidase C, Sphingomonas capsulata prolyl endopeptidase, and the like, for example a proline specific protease set forth herein may be combined with Hordeum vulgare endopeptidase B (EPB2). A “combination”, as used herein, refers to two or more proteases that can be administered contemporaneously in separate formulations, or can be co-formulated in accordance with the invention. In some embodiments the protease or combination of proteases is ingested by an individual contemporaneously with food, e.g. at meal time.

In a fourth aspect, the present invention provides pharmaceutical formulations and unit dose forms suitable for oral administration and containing a protease or combination of proteases as provided by the invention, in an isolated, purified, or recombinant form admixed with one or more pharmaceutically acceptable excipients. Suitable excipients include those disclosed in PCT Publication Nos. 2007/044906; 2008/115411; 2010/021752; and 2010/042203, each of which is incorporated herein by reference.

In a fifth aspect, the present invention provides a method for treating gluten intolerance in a patient in need of such treatment, wherein said treatment reduces the exposure of said patient to immunogenic gluten peptides, said method comprising the step of orally administering to said patient a therapeutically effective dose of a protease of the invention in an isolated, purified, or recombinant form, or a combination of proteases that comprises at least one protease of the invention, or a pharmaceutical formulation thereof contemporaneously with the ingestion of a food that may contain gluten. In one embodiment, the patient has celiac disease. In other embodiment, the patient has dermatitis herpetiformis. In another embodiment, the patient has not been diagnosed as having gluten intolerance but simply prefers not to consume gluten or has gluten sensitivity.

These and other aspects and embodiments of the invention are described in more detail below.

### BRIEF DESCRIPTION OF THE DRAWINGS

FIG. 1. Alignment of sequences and determination of consensus sequence. Asterisks indicate strictly conserved positions and colons and periods indicate full conservation of strong and weak groups, respectively below the multiple sequence alignment. Aligned sequences are (SEQ ID NO:7) A5CG76 from Haemonchus contortus; (SEQ ID NO:8) NP_{—}501599.1 from Caenorhabditis elegans; (SEQ ID NO:9) EFN71004 from Camponotus floridanus; (SEQ ID NO:10) EFN78125 from Harpegnathos saltator; (SEQ ID NO:3) XP_{—}972061; (SEQ ID NO:11) XP_{—}002740665 from Saccoglossus kowalevskii; (SEQ ID NO:1) XP_{—}971305; (SEQ ID NO:2) XP_{—}972807.

### DETAILED DESCRIPTION OF THE EMBODIMENTS

The present invention provides gluten-degrading proteases in isolated, purified, and/or recombinant form. Some of the favorable properties of these proteases with respect to degrading gluten in the gastrointestinal tract include: resistance to degradation by proteases in the gastrointestinal (GI) tract providing longer duration of activity in the GI tract; broad substrate size tolerance that enables degradation of immunogenic gluten peptides regardless of the size of the peptide or protein in which they may be located; synergy with proteases in gluten-degrading activity; broad pH stability and activity range that facilitates optimal activity under acidic gastric conditions; favorable kinetics enabling degradation of gluten before gastric emptying occurs; and low Kₘ for gluten enabling gluten degradation even at low gluten concentrations.

In some embodiments of the invention, a glutenase of the invention is derived from a flour beetle, e.g. members of the darkling beetle genera Tribolium or Tenebrio, which are pests of cereal silos. Flour beetles of interest include, without limitation, Tribolium castaneum (red flour beetle); Tribolium confusum (confused flour beetle); Tribolium destructor (destructive flour beetle); Tenebrio molitor (mealworm beetle); Tenebrio obscurus; etc. Reference may be made to descriptions of flour beetle proteases, e.g. Vinokurov et al. (2009) Arch Insect Biochem Physiol. 70(4):254-79; Goptar et al. (2008) Bioorg Khim. 34(3):310-6; Oppert et al. (2006) Bull Entomol Res. 96(2):167-72; Oppert et al. (2005) Comp Biochem Physiol C Toxicol Pharmacol.; and Liang et al. (1991) FEBS Lett. 278(2):139-42, each specifically incorporated herein by reference.

The amino acid sequences of exemplary proteases are listed by reference to SEQ ID NO and other identifying information in Table 1, and in the sequence listing as proteins (SEQ ID NO:1-3) and encoding nucleotide sequences (SEQ ID NO:4-6). The sequence listing provides the protease amino acid sequence, and in addition a sequence composed of six histidines (6×his tag) and a thrombin cleavage site (LVPRGS) is shown at the C-termini of each protease to illustrate one example of a form of the recombinant proteases of the invention. This optional additional sequence facilitates purification using metal affinity chromatography of the recombinant protease containing them. The nucleotide sequences have been modified from the native sequence to be optimized for expression in Pichia pastoris (SEQ ID NO:4-6) and Escherichia coli (SEQ ID NO: 15,16). Regions of the sequences contain restriction sites introduced by recombinant DNA technology (XhoI on 5′ and KpnI on 3′ end) to facilitate cloning into a Pichia pastoris expression vector in SEQ ID NO 4-6or (NcoI on 5′ and BamHI on 3′ end) to facilitate cloning into an E. coli expression vector in SEQ ID NO 15-16.

TABLE 1
Examples of Proline specific proteases from insects.

| SEQ ID NO | Pubmed Protein ID/Gene ID | Similarity to |
|---|---|---|
| 1, 4, 15 | XP_971305/LOC659946 | Prolylcarboxypeptidase |
| 2, 5, 16 | XP_972807/LOC661563 | Prolylcarboxypeptidase |
| 3, 6 | XP_972061/LOC660762 | Prolylcarboxypeptidase, |
| | | thymus-specific |
| | | serine protease |
| | XP_970931/LOC659540 | Acylpeptide hydrolase |
| | XP_972016/LOC660714 | dipeptidyl-peptidase |

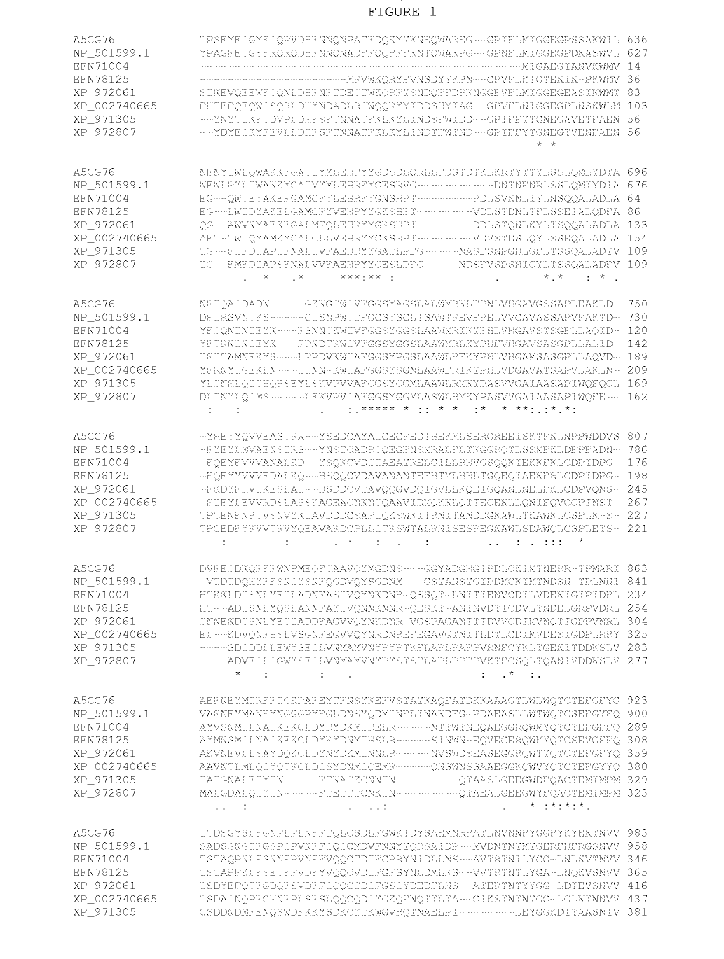

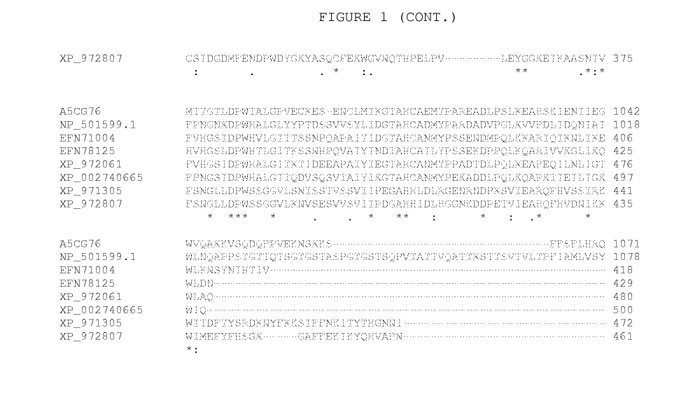

As used herein, a proline specific protease is a protease shown in Table 1 or a protease derived from a eukaryotic cell that has homology to a protease shown in Table 1, or a variant of either. Also optionally included are the proteases set forth in SEQ ID NO:9-11. In one embodiment, the protease is an insect-derived protease, e.g. a flour beetle protease. Thus, the invention provides, in addition to the specific sequences set forth in Table 1, variants, homologs and orthologs of the provided sequences. A variant can be substantially similar to a native sequence, i.e. differing by at least one amino acid, and can differ by at least two but usually not more than about ten amino acids (the number of differences depending on the size of the native sequence). The sequence changes may be substitutions, insertions or deletions. Scanning mutations that systematically introduce alanine, or other residues, may be used to determine key amino acids to be maintained in variant sequences. Homologs or orthologs of the provided sequences include the counterpart proteases in any one of the flour beetles, and will usually have at least about 50% sequence identity at the amino acid level, at least about 75% sequence identity, at least about 80% sequence identity, at least about 85% sequence identity, at least about 90% sequence identity, at least about 95% sequence identity, at least about 99% sequence identity, or more. In various embodiments, a protease of the invention is any protease defined by a consensus sequence based on multiple alignment of several homologs from various organisms is provided below. The multiple sequence alignment was performed using ClustalW2, a general purpose multiple sequence alignment program and is shown in FIG. 1, where the consensus residues are marked at the bottom of the alignment.

Conservative amino acid substitutions that can be used to provide a variant sequence of the invention typically include substitutions within the following groups: (glycine, alanine); (valine, isoleucine, leucine); (aspartic acid, glutamic acid); (asparagine, glutamine); (serine, threonine); (lysine, arginine); and (phenylalanine, tyrosine). Homologs or orthologs of the provided sequences include the counterpart proteases in any one of the flour beetles, and will usually have at least about 50% sequence similarity at the amino acid level, at least about 75% sequence similarity, at least about 80% sequence similarity, at least about 85% sequence similarity, at least about 90% sequence similarity, at least about 95% sequence similarity, at least about 99% sequence similarity, or more.

The amino acid sequence of a naturally occurring protease can be altered in various ways known in the art to generate targeted changes in sequence and so provide variant sequences of the invention. Such variants will typically be functionally-preserved variants, which differ, usually in sequence, from the corresponding native or parent protein but still retain the desired or exhibit enhanced biological activity and/or function. Various methods known in the art can be used to generate targeted changes, e.g. phage display in combination with random and targeted mutations, introduction of scanning mutations, and the like, and provide a variant sequence of the invention. Included are the addition of His or epitope tags to aid in purification, as exemplified herein. Enzymes modified to provide for a specific characteristic of interest may be further modified, for e.g. by mutagenesis, exon shuffling, etc., as known in the art, followed by screening or selection, so as to optimize or restore the activity of the enzyme, e.g. to wild-type levels, and so provide other variant sequences of the invention.

The term “protease” also includes biologically active fragments. Fragments of interest include fragments of at least about 20 contiguous amino acids, more usually at least about 50 contiguous amino acids, and may comprise 100 or more amino acids, up to the complete protein, and may extend further to comprise additional sequences. In each case, the key criterion is whether the fragment retains the ability to digest toxic gluten oligopeptides.

Modifications of interest to the protease that do not alter primary sequence but provide other variant proteases of the invention include chemical derivatization of proteins, including, for example, acylation with, e.g. lauryl, stearyl, myrsityl, decyl, or other groups; PEGylation, esterification; and/or amidation. Such modifications may be used to increase the resistance of the enzyme toward proteolysis, e.g. by attachment of PEG sidechains or lauryl groups to surface lysines. Also included are modifications of glycosylation, e.g. those made by modifying the glycosylation patterns of a protein during its synthesis and processing or in further processing steps; e.g. by exposing the protein to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Another form of modification that does not alter primary sequence is cleavage or removal of sequences that are not required for activity, as well as the removal of sequences that have to be removed before the protease is active. A well known example of the latter type of modification is zymogen activation. Zymogens are inactive forms of proteases that are converted to the active protease by proteolytic cleavage. Thus, in accordance with the invention, proteases can be used as zymogens, so long as the zymogens are activated at the site of action (i.e., in the saliva or stomach) or are preactivated prior to or contemporaneously before contacting them with a gluten-containing food. For example, as zymogen form of a protease may be used to facilitate production or processing, and then, prior to use, be subjected to treatment such that the pro-peptide region of the zymogen is cleaved (and optionally purified away from the active protease). Such pre-activation of a zymogen form may be employed, e.g., to simplify the dosing formulation and/or to reduce the need for activation at the site of action.

Also useful in the practice of and provided by the present invention are proteins that have been modified using molecular biological techniques and/or chemistry so as to improve their resistance to proteolytic degradation and/or to acidic conditions such as those found in the stomach, and to optimize solubility properties or to render them more suitable as a therapeutic agent. For example, the backbone of the peptidase can be cyclized to enhance stability (see Friedler et al. (2000) J. Biol. Chem. 275:23783-23789). Analogs of such proteins include those containing residues other than naturally occurring L-amino acids, e.g. D-amino acids or non-naturally occurring synthetic amino acids.

A proline specific protease of the invention includes any eukaryotic enzyme, including but not limited to a recombinant or purified form of an insect protease, e.g. a flour beetle protease, having a kcat/Km of at least about 2.5 s⁻¹ N/1⁻¹, usually at least about 250 s⁻¹ N/1⁻¹ and preferably at least about 25000 s⁻¹ N/1⁻¹ for cleavage of a gluten oligopeptide that is immunogenic to a celiac disease patient, particularly of longer, physiologically generated peptides, for example the 33-mer from alpha-gliadin, (SEQ ID NO:12) LQLQPF(PQPQLPY)₃PQPQPF, and the 26-mer from gamma-gliadin, (SEQ ID NO:13) FLQPQQPFPQQPQQPYPQQPQQPFPQ. A protease of the invention includes peptidase or protease having a specificity kcat/Km>2 mN/1⁻¹s⁻¹ for the quenched fluorogenic substrate (SEQ ID NO:14) Abz-QPQQP-Tyr(NO₂)-D. These assays can be monitored by HPLC or fluorescence spectroscopy. For the latter assays, suitable fluorophores can be attached to the amino- and carboxy-termini of the peptides.

A protease useful in the practice of the present invention can be identified by its ability to cleave a pretreated substrate to remove toxic (“toxic” as used herein means capable of generating a harmful immune reaction in a celiac disease patient) gluten oligopeptides, where a “pretreated substrate” is a gliadin, hordein, secalin or avenin protein that has been treated with physiological quantities of gastric and pancreatic proteases, including pepsin (1:100 mass ratio), trypsin (1:100), chymotrypsin (1:100), elastase (1:500), and carboxypeptidases A and B (1:100). Pepsin digestion may be performed at pH 2 for 20 min., to mimic gastric digestion, followed by further treatment of the reaction mixture with trypsin, chymotrypsin, elastase and carboxypeptidase at pH 7 for 1 hour, to mimic duodenal digestion by secreted pancreatic enzymes. The pretreated substrate comprises oligopeptides resistant to digestion, e.g. under physiological conditions. A glutenase may catalyze cleavage of pepsin-trypsin-chymotrypsin-elastase-carboxypeptidase (PTCEC) treated gluten such that less than 10% of the products are longer than PQPQLPYPQ (as judged by longer retention times on a C18 reverse phase HPLC column monitored at A₂₁₅). Glutenase assays suitable for characterizing proteases of the invention are also described in U.S. Pat. Nos. 7,303,871; 7,320,788; and 7,534,426, each of which is incorporated herein by reference.

The ability of a protease to cleave a pretreated substrate can be determined by measuring the ability of an enzyme to increase the concentration of free NH₂-termini in a reaction mixture containing 1 mg/ml pretreated substrate and 10 μg/ml of the peptidase or protease, incubated at 37° C. for 1 hour. A protease useful in the practice of the present invention will increase the concentration of the free amino termini under such conditions, usually by at least about 25%, more usually by at least about 50%, and preferably by at least about 100%. A protease includes an enzyme capable of reducing the residual molar concentration of oligopeptides greater than about 1000 Da in a 1 mg/ml “pretreated substrate” after a 1 hour incubation with 10 μg/ml of the enzyme by at least about 2-fold, usually by at least about 5-fold, and preferably by at least about 10-fold. The concentration of such oligopeptides can be estimated by methods known in the art, for example size exclusion chromatography and the like.

A protease of the invention includes an enzyme capable of detoxification of whole gluten, as monitored by polyclonal T cell lines derived from intestinal biopsies of celiac patients; detoxification of whole gluten as monitored by LC-MS-MS; and/or detoxification of whole gluten as monitored by ELISA assays using monoclonal antibodies capable of recognizing sequences specific to gliadin. A protease of the invention may also include an enzyme that reduces the anti-tTG antibody response to a “gluten challenge diet” in a celiac disease patient by at least about 2-fold, more usually by at least about 5-fold, and preferably by at least about 10-fold. A “gluten challenge diet” is defined as the intake of 100 g bread per day for 3 days by an adult celiac disease patient previously on a gluten-free diet. The anti-tTG antibody response can be measured in peripheral blood using standard clinical diagnostic procedures, as known in the art.

The proteases useful in the practice of the present invention may also be isolated and purified in accordance with conventional methods from recombinant production systems and from natural sources. Protease production can be achieved using established host-vector systems in organisms such as E. coli, S. cerevisiae, P. pastoris, Lactobacilli, Bacilli and Aspergilli. Integrative or self-replicative vectors may be used for this purpose. In some of these hosts, the protease is expressed as an intracellular protein and subsequently purified, whereas in other hosts the enzyme is secreted into the extracellular medium. Purification of the protein can be performed by a combination of ion exchange chromatography, Ni-affinity chromatography (or some alternative chromatographic procedure), hydrophobic interaction chromatography, and/or other purification techniques. Typically, the compositions used in the practice of the invention will comprise at least 20% by weight of the desired product, more usually at least about 50% by weight, preferably at least about 85% by weight, at least about 90%, and for therapeutic purposes, may be at least about 95% by weight, in relation to contaminants related to the method of preparation of the product and its purification. Usually, the percentages will be based upon total protein. Proteins in such compositions may be present at a concentration of at least about 500 ug/ml; at least about 1 mg/mg; at least about 5 mg/ml; at least about 10 mg/ml, or more. Suitable methods include those described in PCT Pub. No. 2008/115428, incorporated herein by reference.

In one aspect, the present invention provides a purified preparation of an insect-derived protease. Such enzymes may be isolated from natural sources, but the present invention allows them to be produced by recombinant methods. In one embodiment, such methods utilize a bacterial host for expression, although fungal and eukaryotic systems, including insect systems, find use for some purposes. Coding sequences that contain a signal sequence, or that are engineered to contain a signal sequence can be secreted into the periplasmic space of a bacterial host. An osmotic shock protocol can then be used to release the periplasmic proteins into the supernatant.

Where the enzyme is a cytoplasmic enzyme, a signal sequence can be introduced for periplasmic secretion, or the enzyme can be isolated from a cytoplasmic lysate. Methods for purification include Ni-NTA affinity purification, e.g. in combination with introduction of a histidine tag; and chromatography methods known in the art, e.g. cation exchange, anion exchange, gel filtration, HPLC, FPLC, and the like.

For various purposes, such as stable storage, the enzyme may be lyophilized.

Lyophilization is preferably performed on an initially concentrated preparation, e.g. of at least about 1 mg/ml. Peg may be added to improve the enzyme stability. It has been found that MX PEP can be lyophilized without loss of specific activity. The lyophilized enzyme and excipients is useful in the production of enteric-coated capsules or tablets, e.g. a single capsule or tablet may contain at least about 1 mg. enzyme, usually at least about 10 mg enzyme, and may contain at least 100 mg enzyme, at least about 500 mg enzyme, or more. Coatings may be applied, where a substantial fraction of the activity is retained, and is stable for at least about 1 month at 4° C.

For purposes of combinations of enzymes, the following non-limiting list of proteases is of interest: Hordeum vulgare endoprotease (Genbank accession U19384); X-Pro dipeptidase from Aspergillus oryzae (GenBank ID#BD191984); carboxypeptidase from Aspergillus saitoi (GenBank ID#D25288); Flavobacterium meningosepticum PEP (Genbank ID #D10980); Sphingomonas capsulata PEP (Genbank ID#AB010298); Penicillium citrinum PEP (Genbank ID#D25535); Lactobacillus helveticus PEP (Genbank ID#321529); and Myxococcus xanthus PEP (Genbank ID#AF127082). Combinations of interest include two or more insect-derived proteases. In other embodiments the insect-derived protease may be combined with a non-insect-derived protease, e.g. Hordeum vulgarum endopeptidase C, Sphingomonas capsulata prolyl endopeptidase, and the like, for example a proline specific protease set forth herein may be combined with Hordeum vulgare endopeptidase B (EPB2), and the like. By combination, it is intended that a plurality of proteases are administered contemporaneously in separate formulations, or are co-formulated. In some embodiments the protease or combination of proteases is ingested by an individual contemporaneously with food, e.g. at meal time. The proline- and glutamine-specific proteases described in U.S. Pat. Nos. 7,303,871 and 7,320,788 and in PCT Pub. Nos. 2010/047733, 2009/075816, and 2008/115411, each of which is incorporated herein by reference are especially suitable for us in such combinations. Other glutamine-specific proteases suitable for use in the combination formulations of the invention are described in co-pending U.S. provisional patent application filed herewith at even date by inventor Pawan Kumar and entitled “Proteases for Degrading Gluten” (Attorney Docket No. ALVN-010PRV2), incorporated herein by reference.

The proline-specific gluten degrading proteases of the invention provide certain advantages. They are derived from or highly homologous to proteases that naturally reside in the acidic part of the insect digestive system and so their functional pH range is in an acidic range, making them ideal for degrading gluten in the human stomach. The proteases are proteolytically stable to other insect digestive proteases, and because many insect digestive proteases are homologous to human digestive proteases, this property of proteolytic resistance applies to human digestive proteases. The proteases, in their natural environment, have to break down proteins before a meal is excreted and so have favorable kinetics for meal digestion. Many grains use gluten as a storage protein and the proteases of the invention have evolved to breakdown gluten specifically. Gluten is rich in glutamine and proline residues, and certain of the proteases of the invention are proline specific. These proline specific proteases can be combined, in accordance with the present invention, with glutamine-specific proteases, such as the barley EPB2 protease or its recombinant form ALV001, to make highly potent, gluten-degrading mixtures of proteases.

The methods of the invention, as well as tests to determine their efficacy in a particular patient or application, can be carried out in accordance with the teachings herein using procedures standard in the art. Thus, the practice of the present invention may employ conventional techniques of molecular biology (including recombinant techniques), microbiology, cell biology, biochemistry and immunology within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

For the purposes of the present invention, toxic gliadin oligopeptides are peptides derived during normal human digestion of gliadins and related storage proteins from dietary cereals, e.g. wheat, rye, barley, and the like, that are immunogenic in celiac disease patients, e.g., act as antigens for T cells. Immunogenic peptides are usually from about 8 to 20 amino acids in length, more usually from about 10 to 18 amino acids or longer. Such peptides may include PXP motifs. Determination of whether an oligopeptide is immunogenic for a particular patient is readily determined by standard T cell activation and other assays known to those of skill in the art. Determination of whether a candidate enzyme will digest a toxic gluten oligopeptide can be empirically determined. For example, a candidate may be combined with an oligopeptide or with a pretreated substrate comprising one or more of gliadin, hordein, secalin or avenin proteins that have been treated with physiological quantities of gastric and pancreatic proteases. In each instance, it is determined whether the enzyme is capable of cleaving the oligopeptide. The oligopeptide or protein substrates for such assays may be prepared in accordance with conventional techniques, such as synthesis, recombinant techniques, isolation from natural sources, or the like. For example, solid-phase peptide synthesis involves the successive addition of amino acids to create a linear peptide chain (see Merrifield (1963) J. Am. Chem. Soc. 85:2149-2154). Recombinant DNA technology can also be used to produce the peptide.

The level of digestion of the toxic oligopeptide can be compared to a baseline value.

Gluten becomes much less toxic when it is degraded to peptides shorter than 10 amino acids in length, such as peptides of 8 amino acids, peptides of 6 amino acids, or shorter peptides. The disappearance of the starting material and/or the presence of digestion products can be monitored by conventional methods in model systems, including in vitro and in vivo assay systems. For example, a detectable marker can be conjugated to a peptide, and the change in molecular weight associated with the marker is then determined, e.g. acid precipitation, molecular weight exclusion, and the like. The baseline value can be a value for a control sample or a statistical value that is representative a control population. Various controls can be conducted to ensure that an observed activity is authentic, including running parallel reactions, positive and negative controls, dose response, and the like.

The present invention also provides recombinant nucleic acids comprising coding sequences for the recombinant proteases of the invention. These recombinant nucleic acids include those with nucleotide sequences comprising one or more codons optimized for expression in Pichia pastoris, E. coli, or other host cells heterologous to the cells in which such proteins (or their variants) are naturally produced. Examples of optimized nucleotide sequences are provided in the sequence listing as SEQ ID NO:4-6.

The present invention also provides recombinant expressing vectors comprising nucleic acids encoding the proteases of the invention operably linked to a promoter positioned to drive expression of the coding sequence in a host cell. The present invention also provides methods for producing the proteases of the invention comprising culturing a host cell comprising an expression vector of the invention under conditions suitable for expression of the protease.

As used herein, compounds which are “commercially available” may be obtained from commercial sources including but not limited to Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N. H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.), Novabiochem and Argonaut Technology.

Compounds useful for co-administration with the proteases and treated foodstuffs of the invention can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. 0. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

The proteases of the invention and/or the compounds and combinations of enzymes administered therewith are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the protease and/or other compounds can be achieved in various ways, usually by oral administration. The protease and/or other compounds may be systemic after administration or may be localized by virtue of the formulation, or by the use of an implant that acts to retain the active dose at the site of implantation.

In pharmaceutical dosage forms, the protease and/or other compounds may be administered in the form of their pharmaceutically acceptable salts, or they may also be used alone or in appropriate association, as well as in combination with other pharmaceutically active compounds. The agents may be combined, as previously described, to provide a cocktail of proteolytic activities. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Gluten detoxification for a gluten sensitive individual can commence as soon as food enters the stomach, because the acidic environment (˜pH 2-4) of the stomach favors gluten solubilization. Introduction of a protease into the stomach may synergize with the action of pepsin, leading to accelerated destruction of toxic peptides upon entry of gluten in the small intestines of celiac patients. Such proteases may not require enteric formulation.

In another embodiment, the protease is admixed with food, or used to pre-treat foodstuffs containing glutens. Protease mixed in foods can be enzymatically active prior to or during ingestion, and may be encapsulated or otherwise treated to control the timing of activity. Alternatively, the protease may be encapsulated to achieve a timed release after ingestion, e.g. a predetermined period of time after ingestion and/or a predetermined location in the intestinal tract.

Formulations are typically provided in a unit dosage form, where the term “unit dosage form,” refers to physically discrete units suitable as unitary dosages for human subjects, each unit containing a predetermined quantity of protease in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Depending on the patient and condition being treated and on the administration route, the protease may be administered in dosages of 0.01 mg to 500 mg/kg body weight per day, e.g. about 1-100 mg/kg body weight/per day, e.g., 20 mg/kg body weight/day for an average person. Efficient proteolysis of gluten in vivo for an adult may require at least about 500 units of a therapeutically efficacious enzyme, or at least about 5000 units, or at least about 50,000 units, at least about 500,000 units, or more, for example, about 5×10⁶ units or more, where one unit is defined as the amount of enzyme required to hydrolyze 1 μmol of a chosen substrate per min under specified conditions. It will be understood by those of skill in the art that the dose can be raised, but that additional benefits may not be obtained by exceeding the useful dosage. Those of skill in the art will appreciate that the orally administered proteases of the invention are non-toxic, so the amount of protease administered can exceed the dose sufficient to degrade a substantial amount (e.g., 50% or more, such as 90% or 99%) or all of the gluten in the food with which it is consumed. Dosages will be appropriately adjusted for pediatric formulation. In children the effective dose may be lower. In combination therapy, a comparable dose of the two enzymes may be given; however, the ratio may be influenced by e.g., synergy in activity and/or the relative stability of the two enzymes toward gastric and duodenal inactivation.

Protease treatment of celiac disease or other form of gluten intolerance is expected to be most efficacious when administered before or with meals. However, since food can reside in the stomach for 0.5-2 h, the protease could also be administered up to within 1 hour after a meal. In some embodiments of the invention, formulations comprise a cocktail of selected proteases, for example a combination of a protease of the invention with one or more of Sphingomonas capsulata PEP, Hordeum vulgare cysteine endoprotease B, and the like. Such combinations may achieve a greater therapeutic efficacy.

Those of skill will readily appreciate that dose levels can vary as a function of the specific enzyme, the severity of the symptoms and the susceptibility of the subject to side effects. Some of the proteases are more potent than others. Preferred dosages for a given enzyme are readily determinable by those of skill in the art by a variety of means. A preferred means is to measure the physiological potency of a given compound.

The compositions of the invention can be used for prophylactic as well as therapeutic purposes. As used herein, the term “treating” refers both to the prevention of disease and the treatment of a disease or a pre-existing condition and more generally refers to the prevention of gluten ingestion from having a toxic effect on the patient or reducing the toxicity, relative to the toxic effect of ingestion of the same amount of gluten in the absence of protease therapy. The invention provides a significant advance in the treatment of ongoing disease, and helps to stabilize and/or improve the clinical symptoms of the patient. Such treatment is desirably performed prior to loss of function in the affected tissues but can also help to restore lost function or prevent further loss of function. Evidence of therapeutic effect may be any diminution in the severity of disease, particularly as measured by the severity of symptoms such as fatigue, chronic diarrhea, malabsorption of nutrients, weight loss, abdominal distension, anemia, skin rash, and other symptoms of celiac disease and/or dermatitis herpetiformis and/or gluten sensitivity. Other disease indicia include the presence of antibodies specific for glutens, the presence of antibodies specific for tissue transglutaminase, the presence of pro-inflammatory T cells and cytokines, damage to the villus structure of the small intestine as evidenced by histological or other examination, enhanced intestinal permeability, and the like.

Patients that may be treated by the methods of the invention include those diagnosed with celiac disease or other gluten intolerance through one or more of serological tests, e.g. anti-gliadin antibodies, anti-transglutaminase antibodies, anti-endomysial antibodies; endoscopic evaluation, e.g. to identify celiac lesions; histological assessment of small intestinal mucosa, e.g. to detect villous atrophy, crypt hyperplasia, infiltration of intra-epithelial lymphocytes; and any GI symptoms dependent on inclusion of gluten in the diet.

Given the safety of oral proteases, they also find a prophylactic use in high-risk populations, such as Type I diabetics, family members of diagnosed celiac disease patients, dermatitis herpetiformis patients, HLA-DQ2 positive individuals, and/or patients with gluten-associated symptoms that have not yet undergone formal diagnosis. Such patients may be treated with regular-dose or low-dose (10-50% of the regular dose) enzyme. Similarly, temporary high-dose use of such an agent is also anticipated for patients recovering from gluten-mediated enteropathy in whom gut function has not yet returned to normal, for example as judged by fecal fat excretion assays.

Patients that can benefit from the present invention may be of any age and include adults and children. Children in particular benefit from prophylactic treatment, as prevention of early exposure to toxic gluten peptides can prevent initial development of the disease. Children suitable for prophylaxis can be identified by genetic testing for predisposition, e.g. by HLA typing, by family history, by T cell assay, or by other medical means. As is known in the art, dosages may be adjusted for pediatric use.

The therapeutic effect can be measured in terms of clinical outcome or can be determined by immunological or biochemical tests. Suppression of the deleterious T-cell activity can be measured by enumeration of reactive Th1 cells, by quantitating the release of cytokines at the sites of lesions, or using other assays for the presence of autoimmune T cells known in the art. Alternatively, one can look for a reduction in symptoms of a disease.

Various methods for administration may be employed, preferably using oral administration, for example with meals. The dosage of the therapeutic formulation will vary widely, depending upon the nature of the disease, the frequency of administration, the manner of administration, the clearance of the agent from the host, and the like. The initial dose can be larger, followed by smaller maintenance doses. The dose can be administered as infrequently as weekly or biweekly, or more often fractionated into smaller doses and administered daily, with meals, semi-weekly, or otherwise as needed to maintain an effective dosage level.

The various aspects and embodiments of the invention are illustrated without limitation in the following examples.

### EXAMPLES

The following examples are put forth so as to provide those of ordinary skill in the art with a complete disclosure and description of how to make and use the present invention, and are not intended to limit the scope of the invention or to represent that the experiments below are all or the only experiments performed. Efforts have been made to ensure accuracy with respect to numbers used (e.g., amounts, temperature, and the like), but some experimental errors and deviations may be present. Unless indicated otherwise, parts are parts by weight, molecular weight is weight average molecular weight, temperature is in degrees Centigrade, and pressure is at or near atmospheric.

Favorable properties for proteases for degrading gluten in digestive setting include resistance to other proteases present in the digestive tract to enable longer endurance of enzymes; broad specificity towards peptide size to enable gluten degradation to smallest possible fragments and also to facilitate synergy in two proteases if a combination of enzymes is used; broad pH stability and operating range to enable enzymes to function under acidic gastric conditions; favorable kinetics to enable degradation of majority of gluten before gastric emptying; and a low Kₘ for gluten to enable gluten degradation without significant retardation of gluten degradation rates at low gluten concentrations.

Enzymes have evolved with the above characteristics in several natural sources, including insects that derive proteins from dried grain products, e.g. flour beetles. Flour beetles include Tribolium castaneum (red flour beetle), Tribolium confusum (confused flour beetle); Tribolium destructor (destructive flour beetle); Tenebrio molitor (mealworm beetle); Tenebrio obscurus; and the like. A flour beetle gluten degrading enzyme has one or more of the following advantages: the part of the insect digestive system in which they act is acidic, therefore, functional pH range of the digestive enzymes is acidic; the enzymes are proteolytically stable, relative to other proteases, to other insect digestive proteases, because they have evolved to function in the presence of each other. Because many digestive proteases in insect are homologous to human digestive proteases, the proteolytic resistance property is transferrable to human digestive setting. The digestive proteases have to break down proteins fast enough before the meal is excreted, so the enzymes have favorable kinetics for meal digestion. Many grains have gluten as a storage protein and therefore the digestive enzymes have evolved in an environment in which breakdown of gluten is advantageous to the insect. Because gluten is rich in glutamine and proline residues, these digestive enzyme proteases are efficient in cleaving glutamine and/or proline rich proteins.

T. castaneum's genome was published recently (Nature, 452(7190):949-55, 2008) and >200 putative proteases were identified in the genome. Protease sequences have been catalogued and have been assigned a putative function based on comparison with proteases of known function. Similarly, for T. molitor, the larval midgut cDNA transcripts were analyzed and proteases expressed in the larval midgut were identified and catalogued (Insect Molecular Biology (2007) 16 (4), 455-468). In accordance with the invention, several of these proteases were selected as proline specific glutenases by homology to proline specific proteases These proteases are listed in Table 1 above, and in the sequence listing.

Cloning and expression of proline specific proteases in Pichia pastoris: Codon optimized nucleotide sequences (SEQ ID NO: 4-6) were synthesized and cloned into pPINK-HC vector (Invitrogen) between XhoI and KpnI with a-mating factor sequences appended to the N-terminal of each protease for secreted expression in Pichia pastoris strain 1 using the PichiaPINK® kit (Invitrogen). The a-mating factor secretion signal is removed upon secretion of the protein. The electrocompetent cells were prepared and transformed with the expression plasmids. The strains in the PichiaPink kits were ade2 auxotrophs. The expression plasmids contained the ADE2 gene which complemented the adenine auxotrophy. Transformation of the PichiaPink strains with the expression plasmids enables the strain to grow on medium lacking adenine (Ade dropout medium or minimal medium). The transformants were selected on Ade dropout plates and screened for expression of the proteases.

For protein expression, a 10 mL starter culture was grown for 48 hours in Buffered

Glycerol-complex Medium (BMGY) in a 50 mL Falcon® tube at 24-28° C. The starter culture was used to inoculate 500 mL of BMGY in a 2 L shake flask. Cells were grown for 48 hours at 24-28° C. while shaking at 250 rpm. Cells were centrifuged and resuspended in 100 mL of Buffered Methanol-complex Medium (BMMY) complexed with 1% Methanol to induce protein expression under the control of methanol inducible AOX1 promoter. Protein was expressed for 48 hours at 24-28° C. while shaking at 250 rpm with 0.5% Methanol supplementation after 24 hours.

The protease XP_{—}972061 expressed well in this expression system. The fermentation yield was approximately 50-100 mg/L of protease based on the amount of protein after purification.

Purification of proline specific proteases from Pichia pastoris: After expression of the protein, the cells were removed by centrifugation and supernatant was chilled to 2-8° C. The pH of the supernatant was adjusted to 8.5 by addition of 3 mL 1M Tris (pH 8.5) to 30 mL of supernatant. Additionally, monothioglycerol (MTG) was added to supernatant to a final concentration of 2 mM. 3 mL of Ni-Sepharose FF (GE HealthCare), pre-equilibrated in 50 mM Tris, 2 mM MTG (pH 8.5) were added to supernatant. The suspension was shaken at 2-8° C. for 2 hours for batch binding of the protein to the resin. The slurry was packed into a Kontes gravity flow column. The resin bed was washed with 10 mL of 50 mM Tris, 2 mM MTG (pH 8.5). Protein was eluted in 10 mL of 100 mM potassium phosphate, 2 mM MTG and 250 mM imidazole (pH 5.9). Protease was dialyzed in 100 mM potassium phosphate, 2 mM MTG (pH 5.9) to remove imidazole. Glycerol was added to a final concentration of 20%. Protein was flash frozen in liquid nitrogen in 100 μL aliquots. The purity and protein concentration were estimated by SDS-PAGE analysis. The XP_{—}972061 protein was estimated to be >90% pure based on SDS-PAGE analysis.

Scale-up of production process for XP_{—}972061: The fermentation and purification process was scaled up to a 30 L fed batch fermentation. A cell bank was created from the culture plate of Pichia pastoris containing the expression plasmid for XP_{—}972061.

Two flasks containing BMGY media were inoculated with two 1 mL aliquots of cell bank glycerol stock. The flasks were shaken at 28° C. for 48 hrs. The shake flask cultures reached an OD of 54.3 and then were used to inoculate a 30 L fermentor containing BMGY medium. The fermentation was performed at 28° C. with 400-800 rpm agitation and an aeration rate of 20 Ipm air. The pH was controlled at 5.5 using ammonium hydroxide and sulfuric acid. The dissolved oxygen levels were maintained at 30%. The growth was monitored hourly, and the glycerol feed was initiated when the glycerol concentration dropped below 1% and a sudden spike in dissolved oxygen was observed. The glycerol feed was initiated at 5 mL/min and gradually reduced to 0 mL/min over the course of 5 hr. Protein expression was induced by initiating methanol feed at 1.5 mL/min, which was increased to 5 mL/min over the course of 5 hr and continued until the end of fermentation. Fermentation was stopped after 48 hr of induction, and cells were removed by centrifugation. Approximately 19 L of supernatant were collected and frozen at −80° C. for further processing.

A BPG 100 column (GE) was packed with 1.4 L HIS Select Nickel Affinity resin (bed height 18.8 cm). The column was washed with 2 column volumes (CVs) of water and then equilibrated with 4 CVs of equilibration/wash buffer (50 mM Tris containing 2 mM MTG, pH 8.5) at 130 mL/min flow rate using an Akta Pilot chromatography system. The pH of the supernatant from 30 L fermentation was adjusted to 8.5 by 1M Tris (pH 8.5) and loaded onto the column at 130 mL/min. The column was washed with 2 CVs of Equilibration/Wash buffer. The protein was then eluted using elution buffer (100 mM potassium phosphate, 250 mM imidazole, 2 mM MTG, pH 5.6). The elution fraction (1.5 L) was diafiltered into lyophilization buffer (100 mM potassium phosphate, 5 mM EDTA, 2.5% mannitol, 2 mM MTG, pH 5.6). After diafiltration, the product was filtered through a 0.2 micron filter and stored at −80° C. The concentration of the frozen XP_{—}972061 was 7.4 mg/mL. After purification, the product was lyophilized and dispensed in 250 mg aliquots in 30 mL bottles and stored at 2-8 ° C. until use. Prior to use in gluten degradation studies, the product was freshly reconstituted in water to a concentration of 10 mg/mL protein.

Cloning and expression of proline specific proteases in Escherichia coli (E. coli): Codon optimized nucleotide sequences (SEQ ID NO: 15-16) were synthesized and cloned into pET28b vector (Novagen) between NcoI and BamHI sites for the cytosolic expression in E. coli strain BL21 (DE3). The subcloning into E. coli expression vector results into addition of two N-terminal residues “Met Gly” in the appended proteins sequences (SEQ ID NO: 1, 2). The chemical competent cells were prepared and transformed with expression plasmid. The expression plasmids contained the kan+ gene to provide resistance to the antibiotic kanamycin. Transformation of the E. coli strains with the expression plasmids enabled the strain to grow on medium containing kanamycin. The transformants were selected on kanamycin containing plates and screened for expression of the proteases.

For protein expression, a 10 mL starter culture was grown for 12 hours in Luria Broth

(LB) in a 50 mL Falcon tube at 37° C. with shaking at 250 rpm. The starter culture was used to inoculate 1000 mL of LB in a 2 L shake flask. Cells were grown at 37° C. with shaking at 250 rpm to an optical density (OD600) of 0.6-0.8 measured by absorbance at 600 nm. Cells were cooled below 30° C. and Isopropyl β-D-1-thiogalactopyranoside (IPTG) was added to a concentration of 0.2-1 mM to induce protein expression under the control of IPTG inducible T7 promoter. Protein was expressed for 12 hours at 30° C. with shaking at 250 rpm.

Proteases expressed well in this expression system as inclusion bodies (IB). The fermentation yield was approximately 50 mg/L of proteases.

Refolding of proline specific proteases obtained from E. coli: Cells are harvested by centrifugation at 5000×g for 15 minutes. Harvested cells are resuspended in lysis buffer (50 mM Tris, pH 8.5, 2 mM MTG). The cells are lysed by sonication and inclusion bodies are separated from soluble matter by centrifugation at 10,000×g for 30 minutes. For washing, inclusion bodies are resuspended in water to ½ of the original volume. Inclusion bodies are recovered by centrifugation at 10,000×g for 30 minutes. The inclusion body washing process is repeated once. Washed inclusion bodies are solubilized in solubilization buffer (50 mM Tris, pH 8.5, 2 mM MTG, 7 M urea) for 4-6 hours at room temperature in ½ the original volume. After solubilization, insoluble matter is removed by centrifugation at 10,000×g for 30 minutes. Protein refolding is carried out by diluting protein 1 to 20 fold in 10 mM sodium phosphate, pH 8.2, 880 mM arginine, 1 mM GSH (reduced glutathione) and 1 mM GSSG (oxidized glutathione) at 4° C. and incubating overnight.

Degradation of Celiac disease relevant peptides by proline specific proteases: 50 μg/mL of various celiac disease relevant peptides were incubated with 25 μg/mL of XP_{—}972061 at 37 ° C. at pH 4.5 for 1 hr. In this study the three peptide used were PQQPQQSFPQQQPPF, QLPQQPQQF and LGQQQPFPPQQPYPQPQPF. The degradation of peptides was analyzed by reverse phase liquid chromatography (RPLC). Each of the three peptides was completely proteolyzed by XP_{—}972061 as observed by elimination of peptide peak compared to no enzyme control reaction. This suggests that an active protease was purified and that the protease was able to degrade celiac disease relevant peptides at low pH.

Pepsin stability of proteases under low pH conditions: 0.5 mg/mL XP_{—}972061 were incubated with 0.4 mg/mL pepsin and 1 mg/mL BSA at pH 3.0 at 37C. 10 μL of XP_{—}972061 were taken at various timepoints and added to 990 μL of chromogenic substrate H-Ala-Phe-Pro-pNA (0.2 mM substrate in 20 mM sodium phosphate pH 7.0, 10% DMSO) and the activity was monitored at 410 nm by the release of pNA chromophore from substrate by proteolytic action of XP_{—}972061. The data is shown below as percentage of initial activity (˜5500 U/mg for above assay conditions) and indicates that XP_{—}972061 has a half-life of approximately 3 min, demonstrating that XP_{—}972061 has moderate stability to short term exposure to highly concentrated pepsin in low pH environment. The resistance to pepsin in low pH environment is valuable for sustained activity of this protease in diverse gastric environment.

TABLE 3
Stability of XP_972061 against proteolysis by pepsin at pH 3.0

| Time (min) | XP_972061 Activity (%) |
|---|---|
| 0 | 100.0 |
| 2 | 63.0 |
| 4 | 34.2 |
| 5 | Not tested |
| 10 | Not tested |
| 15 | Not tested |
| 20 | Not tested |
| 30 | Not tested |

Stability of proteases under oxidizing conditions: A banana, ˜335 mL 40 mM HCl and one Amy's Gluten Free Korma meal were mixed and incubated at room temperature for 30 min. The solids were removed by centrifugation and the supernatant, which oxidatively inactivates certain cysteine proteases, was used for the following experiments. 0.5 mg/mL of XP_{—}972061 were incubated with 70% of oxidizing meal supernatant at pH 4.0 at 37C. 10 μL of XP_{—}972061 were taken at various timepoints and added to a chromogenic substrate H-Ala-Phe-Pro-pNA and the activity monitored at 410 nm by the release of pNA chromophore from substrate by proteolytic action. The data is shown below and indicates that XP_{—}972061 have a half-life of greater than 30 min, demonstrating that XP_{—}972061 have very high stability to exposure to oxidizing conditions. The resistance to oxidation is valuable for sustained activity in diverse gastric environments.

TABLE 4
Stability of XP_972061 under oxidative conditions

| Time (min) | XP_972061 Activity (%) |
|---|---|
| 0 | 100 |
| 5 | 108.3 |
| 10 | 117.1 |
| 15 | 117.1 |
| 20 | 126.3 |
| 30 | 122.5 |
| 60 | Not tested |

High pH stability of XP_{—}972061: 0.5 mg/mL of XP_{—}972061 was incubated with 250 mM Tris at pH 7.5 at 37° C. 10 μL of XP_{—}972061 were taken at various timepoints and added to a chromogenic substrate H-Ala-Phe-Pro-pNA and the activity monitored at 410 nm by the release of pNA chromophore from substrate by proteolytic action. The data is shown below and indicates that XP_{—}972061 has a half-life of greater than 30 min, demonstrating very high stability to exposure to high pH conditions. The resistance to high pH condition is valuable for sustained activity of these proteases in diverse gastric environments.

TABLE 5
Stability of XP_972061 at high pH condition

| Time (min) | XP_972061 Activity (%) |
|---|---|
| 0 | 100 |
| 3 | 95.9 |
| 5 | 91.5 |
| 10 | 82.7 |
| 20 | 72.8 |
| 30 | 64.6 |
| 60 | 46.2 |

Degradation of pepsin digested gluten: 6 mg/mL (0.6 mg) of pepsin-digested gluten were incubated with 25-100 μg/mL of ALV001 (EP-B2), a glutenase with specificity towards glutamine residues in gluten, 100 μg/mL of XP_{—}972061 or the combination of the two proteases at pH 3.0 and 37 ° C. for 10 minutes in a 100 μL reaction. The gluten degradation was analyzed qualitatively by RPLC and quantitatively by ELISA (data tabulated below), which measures one of the immunostimulatory epitopes relevant to celiac disease. RPLC data qualitatively indicated that XP_{—}972061 alone is able to degrade gluten and that the combination of XP_{—}972061 and ALV001 degraded gluten to a greater extent than either of the proteases alone. Similarly, ELISA data quantitatively indicated that the combination of the two proteases was more effective that either of the proteases alone. The observation that the combination of the two proteases degraded gluten more effectively than twice the amount of ALV001 demonstrates a synergy in gluten degradation between the two proteases.

TABLE 6
Quantitative analysis of degradation of immunostimulatory epitope
in pepsin digested gluten by ALV001 and XP_972061

| ALV001 | XP_972061 | Fold degradation of Gluten |
|---|---|---|
| concentration, | concentration, | relative to no enzyme |
| μg/mL | μg/mL | control |
| 0 | 100 | 5 |
| 25 | 0 | 2 |
| 50 | 0 | 4 |
| 100 | 0 | 9 |
| 25 | 100 | 7 |
| 50 | 100 | 30 |
| 100 | 100 | 63 |

All publications, patents, and patent applications cited in this specification are herein incorporated by reference as if each individual publication, patent, or patent application were specifically and individually indicated to be incorporated by reference.

The present invention has been described in terms of particular embodiments found or proposed by the inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in methods, structures, and compounds without affecting the biological action in kind or amount. All such modifications are intended to be included within the scope of the appended claims.

1. A gluten-degrading protease from an insect that feeds on gluten containing dried grain products, in isolated, purified, or recombinant form.

2. The protease of claim 1, wherein said insect is a flour beetle.

3. The protease of claim 1, wherein said flour beetle is Tribolium castaneum.

4. The protease of claim 1 wherein said protease is a protease set forth in Table 1, or a homolog, ortholog or variant thereof.

5. The protease of claim 4, wherein said homolog, ortholog or variant has at least 80% sequence identity to a protease set forth in Table 1.

6. The protease of claim 4, wherein said protease degrades said gluten in a foodstuff to fragments shorter than 8 amino acids.

7. The protease of claim 4, wherein said protease digests gluten fragments that are resistant to normal digestive enzymes.

8. The protease of claim 1, wherein said protease is formulated with a pharmaceutically acceptable excipient.

9. The protease of claim 1, wherein said protease is admixed with food.

10. The protease according to claim 1, wherein said protease is stable to acid conditions.

11. A recombinant expression vector for a gluten-degrading protease, comprising a coding sequence for a protease set forth in claim 1 and a promoter that drives expression of said protease in a suitable host cell,

12. A method for degrading gluten in food, said method comprising contacting gluten-containing food with one or more proteases of claim 1.

13. A pharmaceutical formulation suitable for oral administration that contains a protease of claim 1 admixed with one or more pharmaceutically acceptable excipients.

14. A pharmaceutical formulation of claim 13, further comprising one or more non-insect proteases.

15. The pharmaceutical formulation of claim 14, wherein said non-insect protease is one or more of Hordeum vulgare endoprotease (Genbank accession U19384); X-Pro dipeptidase from Aspergillus oryzae (GenBank ID#BD191984); carboxypeptidase from Aspergillus saitoi (GenBank ID#D25288); Flavobacterium meningosepticum PEP (Genbank ID #D10980); Sphingomonas capsulata PEP (Genbank ID#AB010298); Penicillium citrinum PEP (Genbank ID#D25535); Lactobacillus helveticus PEP (Genbank ID#321529); and Myxococcus xanthus PEP (Genbank ID#AF127082)

16. A method for treating gluten intolerance in a patient in need of such treatment, wherein said treatment reduces exposure of said patient to immunogenic gluten peptides, said method comprising the step of orally administering to said patient a therapeutically effective dose of one or more proteases of claim 1 or a pharmaceutical formulation thereof contemporaneously with the ingestion of a food that may contain gluten.

17. The method of claim 16, further comprising orally administering to said patient a therapeutically effective dose of one or more non-insect proteases.

18. The method of claim 17, wherein said non-insect protease is one or more of Hordeum vulgare endoprotease (Genbank accession U19384); X-Pro dipeptidase from Aspergillus oryzae (GenBank ID#BD191984); carboxypeptidase from Aspergillus saitoi (GenBank ID#D25288); Flavobacterium meningosepticum PEP (Genbank ID #D10980); Sphingomonas capsulata PEP (Genbank ID#AB010298); Penicillium citrinum PEP (Genbank ID#D25535); Lactobacillus helveticus PEP (Genbank ID#321529); and Myxococcus xanthus PEP (Genbank ID#AF127082)


SEQUENCE LISTING (16 sequences)

SEQ ID NO: 1 - protein, 472 aa, Triboleum castaneum
YNYTTKFIDVPLDHFSFTNNATFKLKYLINDSFWIDDGPIFFYTGNEGAVETFAENTGFIFDIAPTFNALIVFAEHRYYGATLPFGNASFSNPGHLGFLTSSQALADYVYLINHLQTTHQRSEYLSKVPVVAFGGSYGGMLAAWLRMKYPASVVGAIAASAPIWQFQGLTPCENFNRIVSNVYKTAVDDDCSAPIQKSWKIIRNITANDDGKAWLTKAWKLCSPLKSSDIDDLLEWYSEILVNMAMVNYPYPTKFLAPLPAFPVRNFCYKLTGEKITDDKSLVTAIGNALEIYTNFTKATKCNNINQTAASLGEEGWDFQACTEMIMPMCSDDNDMFENQSWDFKKYSDKCYTKWGVRQTNAELPILEYGGKDITAASNIVFSNGLLDPWSSGGVLSNISSTVSSVIIPEGAHHLDLRGENRNDPKSVIEARQFHVSSIRKWITDFYYSRDKNYFKKSIFFNKITYTHGNNI

SEQ ID NO: 2 - protein, 461 aa, Triboleum castaneum
YDYETKYFEVLLDHFSFTNNATFKLKYLINDTFWTNDGPIFFYTGNEGTVENFAENTGFMFDIAPSFNALVVFAEHRYYGESLPFGNDSFVSPSHIGYLTSSQALADFVDLINYLQTMSLEKVPVIAFGGSYGGMLASWLRMKYPASVVGAIAASAPIWQFETPCEDFYKVVTRVYQEAVAKDCPLLITKSWTALRNISESPEGKAWLSDAWQLCSPLETSADVETLIGWYSEILVNMAMVNYPYSTSFLAPLPPFPVKTFCSQLTQANIVDDKSLVMALGDALQIYTNFTETTTCNKINQTAEALGEEGWYFQACTEMIMPMCSIDGDMFENDPWDYGKYASQCFEKWGVNQTHPELPVLEYGGKEIKAASNIVFSNGLLDPWSSGGVLKNVSESVVSVIIPDGAHHIDLRGGNKDDPETVIEARQFHVDNIKKWIMEFYFHSGKGAFFEKIKYQHVARN

SEQ ID NO: 3 - protein, 480 aa, Triboleum castaneum
RIFRNGRMVGGNLGEPKCNCKESSIKEVQEEWFTQNLDHFNPTDETTWKQRFYSNDQFFDPKNGGPVFLMIGGEGEASIKWMTQGAWVNYAEKFGALMFQLEHRYYGKSHPTDDLSTQNLKYLTSQQALADLATFITAMNEKYSLPPDVKWIAFGGSYPGSLAAWLRFKYPHLVHGAMSASGPLLAQVDFKDYFRVIKESLATHSDDCVTAVQQGVDQIGVLLKQEIGQANLNELFKLCDPVQNSINNEKDISNLYETIADDFAGVVQYNKDNRVGSPAGANITIDVVCDIMVNQTIGPPVNRLAKVNEVLLSAYDQKCLDYNYDKMINNLRNVSWDSEASEGGRQWTYQTCTEFGFYQTSDYEPQIFGDQFSVDFFIQQCTDIFGSIYDEDFLNSATERTNTYYGGLDIEVSNVVFVHGSIDPWHALGITKTIDEEAPAIYIEGTAHCANMYPPADTDLPQLKEAREQILNLIGTWLAQ

SEQ ID NO: 4 - DNA, 1476 bp, Tribolium castaneum
ctcgagaaaaggccttataactacaccactaaatttattgatgtgccgttggaccatttctcttttaccaataacgctaccttcaagctgaaatatctgattaatgattccttttggattgatgatggtcctatcttcttttacaccggtaacgaaggtgctgtggaaactttcgcagaaaataccggttttattttcgatattgcgccaacctttaacgctctgattgttttcgccgaacacagatattacggtgcaaccctgccgtttggcaatgcgtcattctccaaccctggtcatctgggctttttgactagttctcaggctctggccgattatgtgtacctgattaatcacctgcaaaccacccatcagcgctccgaatatctgtcaaaggtcccagttgtggcattcggcggtagctacggcggcatgctggcggcttggctgcgtatgaaatatccggcctccgtggttggtgcaattgcggcttctgcccctatctggcaatttcagggtctgaccccatgtgaaaacttcaatagaattgtgtccaacgtgtacaagactgcagttgacgatgattgctcagcgccgattcaaaaatcctggaagattattcgcaatattaccgctaacgatgatggcaaagcctggctgaccaaggcatggaaactgtgttcccctctgaagtcttccgatattgatgatctgttggaatggtattcagagattctggtgaatatggcgatggtcaactacccatatccgaccaaatttctggctcctctgccagccttcccggttcgtaatttttgctacaagttgactggtgaaaaaattaccgatgataagagcctggtgaccgcaattggtaacgctctggaaatctacaccaatttcactaaagctaccaagtgtaacaatattaaccagaccgccgcaagtctgggcgaagaaggttgggattttcaagcgtgcaccgaaatgattatgcctatgtgttctgatgataatgatatgttcgaaaaccagtcctgggattttaaaaagtactcagataaatgctatactaagtggggtgtgagacaaaccaatgcagaactgccaattctggagtacggcggtaaagatattactgccgcttccaacattgttttcagtaatggtctgcttgatccttggtcttccggtggtgtgctgtcaaacatcagcagtaccgtctcttccgtgattattcctgaaggtgcccaccatctggatctgcgcggtgaaaatcgtaacgatccgaagtcagttattgaagctagacagtttcacgttagctctattcgcaaatggattactgatttctattactctcgtgataagaattattttaaaaaatccatctttttcaacaaaattacctacactcatggtaataacattctggttccacgtggaagtcatcaccatcaccatcactaaggtacc

SEQ ID NO: 5 - DNA, 1443 bp, Tribolium castaneum
ctcgagaaaaggccttatgattacgaaaccaaatattttgaagtgttgctggaccatttctcttttactaacaatgctaccttcaagttgaaatacctgattaacgataccttttggaccaatgatggtccgatcttcttttatactggtaacgaaggaacagtggaaaatttcgccgaaaacaccggttttatgttcgatattgcaccttcctttaatgcgttggttgtgttcgctgaacacagatactatggtgaatcactgccatttggcaacgattccttcgtgtccccgtctcatattggttacttgacctcctcacaggccctggcagattttgttgacttgattaattatctgcaaactatgagcttggagaaggtgcctgtgattgcgttcggtggaagttacggtggtatgctggcttcttggctgcgcatgaaatatccagcctccgttgtgggcgcaattgcggcttcagccccgatctggcagtttgaaaccccttgtgaagatttctacaaggtggttacgcgtgtgtatcaagaagcagtggcgaaagattgcccactgctgattaccaagtcctggactgctctgagaaacatttccgaatctccggagggtaaagcctggctgtccgatgcatggcagctgtgttcacctttggagacctccgcggatgttgaaaccctgattggttggtactccgaaattctggtgaatatggctatggtcaactatccatactctacctcctttctggccccgctgcctccattcccggttaagactttttgctcacaactgacccaggcaaatattgtggatgataaatctctggtgatggcgctgggcgatgctctgcaaatctataccaacttcaccgaaactaccacttgtaataagattaaccagaccgccgaggcactgggtgaagaaggttggtactttcaagcgtgcactgaaatgattatgcctatgtgttccattgatggcgatatgttcgaaaatgatccttgggattatggtaaatacgcttctcagtgctttgaaaagtggggtgttaaccaaacccacccggaactgcctgtgctggaatatggcggtaaagaaattaaggccgcatccaatattgtcttctcaaacggtctgcttgatccttggtcctcaggcggtgttctgaaaaatgtgtctgaatctgtggtgtccgttattattccggatggtgcgcatcacatcgatctgcgcggtggtaacaaggatgatcctgaaactgtgatcgaagcacgtcagttccatgttgataacattaaaaagtggatcatggaattttacttccactctggaaagggtgctttctttgaaaagatcaagtaccaacatgttgctagaaacctggttccacgtggaagtcatcaccatcaccatcactaaggtacc

SEQ ID NO: 6 - DNA, 1500 bp, Tribolium castaneum
ctcgagaaaaggcctcgtatttttcgcaacggtcgtatggtgggtggcaatctgggtgaaccgaaatgtaactgcaaagaatcttccattaaagaagtgcaggaagaatggttcacccaaaatctggatcattttaaccctactgatgaaaccacctggaagcagagattctattcaaatgatcaatttttcgatccaaaaaacggtggcccggtttttctgatgattggtggtgaaggtgaggcttctattaagtggatgacccagggtgcctgggtgaattacgcagaaaaattcggtgcgctgatgtttcaattggagcaccgctattacggcaagtcccatcctactgatgatctgtctacacagaacctgaaatatctgacctcccaacaggctttggccgatctggcaaccttcattactgcgatgaatgaaaagtactcactgccaccggatgtgaaatggattgcttttggtggcagctatcctggaagtctggccgcatggctgcgtttcaagtacccacacctggttcatggtgcgatgtctgcttccggtccgctgctggcccaagtggattttaaagattatttcagagtgattaaagaatctctggcaacccactccgatgattgtgttaccgcggtgcagcagggtgttgatcagattggtgttctgctgaaacaagaaattggtcaggctaacctgaatgaactgtttaagctgtgcgatcctgtgcaaaacagtatcaacaacgaaaaggatatctctaacctgtacgaaaccatcgccgatgatttcgcaggcgtggttcagtataacaaggataatcgcgtgggttccccagcgggtgctaacattactattgatgtggtttgtgatattatggtgaatcaaaccattggcccgcctgtgaaccgtctggccaaagttaatgaagtgctgctgtcagcatacgatcagaagtgcttggactataactacgataaaatgattaataacctgagaaatgtgtcctgggattccgaggcgtctgaaggtggccgccaatggacctatcagacctgtactgagtttggcttctaccaaacctccgattatgaaccacaaatcttcggtgatcaattctcagttgatttctttattcagcaatgcaccgatatttttggcagcatttacgatgaagattttctgaactctgctaccgaacgtactaatacctattacggtggcctggatattgaagtgtctaacgttgtgtttgttcatggttctattgatccgtggcacgccctgggtattactaaaaccattgatgaagaagcacctgcgatttatattgaaggtactgctcattgtgccaatatgtacccaccggcagataccgatctgcctcagctgaaagaagccagagaacaaattctgaacctgattggtacttggctggctcagctggttccacgtggaagtcatcaccatcaccatcactaaggtacc

SEQ ID NO: 7 - protein, 1071 aa, Haemonchus contortus
  VARIANT 520, 545, 555, 764, 832: Xaa = Any Amino Acid
MLVSSWMPMLHLGHPMTATNELNLTGFASQGGFGGREAYLKQKLDHTQEVKEWSQRYFYNNRYYRKGGNVAFLMLGGMGVLDIGWVTNEKIPFVQMAKERGALMFALEHRFYGKSRPTDDLSVKNLKYLTIEQAIGDIKTFIEEMNKKHKLENPKWIVFGGSYAGSLALWARDKYKDENLIAGAVASSPIMRPKFDFWEATQFAEKEIQKVDKKCGESIRIGFMQMIDMLGNQVGRSQLSELFKMRPRFLTPDLRNIQLLNSIQLNNFISAVQFRGGPYMQNGTHSYNLKQLCEIMNTETIDQLTALERVSNVRHLQSKYLNDMDKYTPVDFDALMKYLLKKDFDEEGWASVDRASLWQRCTQLGSFPTTDGAINSIFGSLVSIDFYADLCQVFGEKFNAEHIEMTVEETLQHYGGADNYKGTNVVIANGGSDPYHLLSKLSSRDPTVVTYLIEGGSHCGDMFPFEFNNSPTAAPGTKLIHLLTAQNIDTWISGVPSPFHIDKKEPEKQLVKLEVSAMPXTPLPSPSLNGVKSRFSLFKRQSTQXVATQLTPREXDILQRVRLGRPPHGFVPNLDTVDTPSEYETGYFTQPVDHFNNQNPATFDQKYYKNEQWAREGGPIFLMIGGEGPSSAKWILNENYTWLQWAKKFGATTYMLEHRYYGDSDLQRLLFDSTDTKLKRTYTTYLSSLQMLYDTANFIQAIDADNGKKGTWIVFGGSYAGSLALWMRKLFPNLVHGAVGSSAPLEAKLDYHEYYQVVEASIRXYSEDCAYAIGEGFEDIHEKMLSERGREEISKTFKLNPPWDDVSDVFEIDKQFFFWNPMEQFTAAVQYXGDNSGGYADGHGIPDLCKIMTNERRTPMARIAEFNEYMTRFFTGKPAFEYTFNSYKEFVSTAYKAQFATDKKAAAGTLWLWQTCTEFGFYGTTDSGYSLFGNPLPLNFFTQLCSDLFGWKIDYSAEMNRRATLNVNNRYGGRYKYEKTNVVMTYGTLDPWTALGPVECKESENCLMIKGTAHCAEMYPAREADLPSLKEARSKIENIIEGWVQAKKVSQDQRPVEKNSKKSFFSFLHRQ

SEQ ID NO: 8 - protein, 1080 aa, Caenorhabditis elegans
MTRNLLLLTLLVSFVLAIIPNHYHFKKHLKRGSRKYGNSETAMTTGYMAQNLDHLIGNASGTFTQRYLYSQQYTLHQRTAFLYVSGVEGPNVVLDDRTPIVKTAKQFGATIFTLEHRYYGESKPNVDKLDAYNLRHLNSFQATQDVISFIKYANVQFNMDQDVRWVVWGIGYGGIIAAEARKLDPNSVSGVIASSTPLTHEYDFWRFNHRVAIVLAETGGSLCYRKVANGFADIREAMKTPEGRLNISDLFQLNPRLNETALNYNDIQMFYLAIIAPFQEIVEFNDDFDLSIADLCTTIDKSNWTNMEVVYQAYVYLSTTLDGFAGPMDISYQDFVDSLGDQSVDSGWIDNRIWQYQVCTEFGWFYTTNDNEQGLFGPVVPASLFLNQCFDIFPDANLTATGLRDSIINYNNFYGSSYDYSGTNAVFTNGMNDPWRELGKTSTGDFSVVAYLIPDASTASDMFPGNTNNSFIIQAHNLMTENINVWLNGPRNPKTFVNTTVPWTRPYWGEYGSLRETMLKQEVESKFSKLENGRTSEKTFPEPKFKKVFLGRPPHGFLPESDFNMSPDDYPAGFETGSFRQRQDHFNNQNADFFQQRFFKNTQWAKPGGPNFLMIGGEGPDKASWVLNENLPYLIWAKKYGATVYMLEHRFYGESRVGDNTNFNRLSSLQMIYDIADFIRSVNIKSGTSNPWITFGGSYSGLISAWTREVFPELVVGAVASSAPVFAKTDFYEYLMVAENSIRSYNSTCADRIQEGFNSMRALFLTKGGRQTLSSMFKLDPPFADNVTDIDQHYFFSNIYSNFQGDVQYSGDNMGSYANSYGIPDMCKIMTNDSNTPLNNIVAFNEYMANFYNGGGPYFGLDNSYQDMINFLINAKDFGPDAEASLLWTWQTCSEFGYFQSADSGNGIFGSPTPVNFFIQICMDVFNNYYQRSAIDPMVDNTNYMYGERFHFRGSNVVFPNGNKDPWHALGLYYPTDSSVVSYLIDGTAHCADMYPARDADVPGLKVVRDLIDQNIAIWLNQAPPSTGTTQTSGTGSTASPGTGSTSQPVTATTVQATTKSTTSVTVLTPFIAMLVSYLL

SEQ ID NO: 9 - protein, 418 aa, Camponotus floridanus
MIGAEGIANVKWMVEGQWIEYAKEFGAMCFYLEHRFYGNSHPTPDLSVKNLIYLNSQQALADLAYFIQNINIEYKFSNNTKWIVFGGSYGGSLAAWMRIKYPHLVHGAVSTSGPLLAQIDFQEYFVVVANALKDYSQKCVDTIAEAYRELGILLRHVGSQQKIEKKFKLCDPIDPGHTKKLDISNLYETLADNFASIVQYNKDNRQSSQTLNITIENVCDILVDEKIGIPIDRLAYVSNMILNATKEKCLDYRYDKMIRELRNTTWTNEQAEGGRQWMYQTCTEFGFFQTSTAQPNLFSNNFPVNFFVQQCTDIFGPRYNIDLLNSAVTRTNILYGGLNLKVTNVVFVHGSIDPWHVLGITTSSNPQAPAIYIDGTAHCANMYPSSENDMPQLKKARIQIKNLIKEWLKNSYNIHTIV

SEQ ID NO: 10 - protein, 429 aa, Harpegnathos saltator
MRVWKQRYFVNSDYYKPNGPVFLMIGTEKIKPKWMVEGLWIDYAKELGAMCFYVEHRYYGKSHPTVDLSTDNLTFLSSEIALQDFAYFIRNINIEYKFPNDTKWIVFGGSYGGSLAAWMRLKYPHFVHGAVSASGPLLALIDFQEYYVVVEDALKQHSQQCVDAVANANTEFHTMLHHLTGQEQIAEKFRLCDPIDPGHTADISNLYQSLANNFAYIVQNNKNNRQESKTANINVDTICDVLTNDELGRPVDRLAYMNSMILNATKEKCLDYKYDNMIHSLRSINWNEQVEGERQWMYQTCSEVGFFQTSTARPKLFSETFPVDFYVQQCVDIFGPSYNLDMLKSVVTRTNTLYGALNQKVSNVVHVHGSLDPWHTLGITKSSNHPQVAIYINDTAHCAILYPSSEKDPPQLKQARIVVKGLIKQWLDN

SEQ ID NO: 11 - protein, 500 aa, Saccoglossus kowalevskii
MAGSNGMGYLLCVFLTVLPSVFSLPYFMNGRPRGGMVGVPVLSERPHTEPQEQWISQRLDHYNDADLRTWQQRYYIDDSHYIAGGPVFLNIGGEGPLNSKWLMAETTWIQYAMKYGALCLLVEHRYYGKSHPTVDVSTDSLQYLSSEQALADLAYFRNYIGEKLNITNNKWIAFGGSYSGNLAAWFRIKYPHLVDGAVATSAPVLAKLNFTEYLEVVRDSLASSKAGEACNKNIQAAVIDMQKKLQTTEGEKLLQNIFQVCGPINSTELKDVQNFHSLVSGNFEGVVQYNRDNREFEGAVGTNITLDTLCDIMVDESIGDPLHRYAAVNTLMLQTYQTKCLDISYDNMIQEMRQNSWNSSAAEGGKQWVYQTCTEFGYYQTSDAINQPFGHNFPLSFSLQQCQDIYGKQFNQTTLTAGIKSTNTNYGGLGLKTNNVVFPNGSIDPWHALGITQDVSQSVTAIYIKGTAHCANMYPEKADDLPQLKQARKTIEILIGKWIQ

SEQ ID NO: 12 - protein, 33 aa, Triticum aestivum
LQLQPFPQPQLPYPQPQLPYPQPQLPYPQPQPF

SEQ ID NO: 13 - protein, 26 aa, Triticum aestivum
FLQPQQPFPQQPQQPYPQQPQQPFPQ

SEQ ID NO: 14 - protein, 5 aa, Artificial Sequence
  synthetic peptide
QPQQP

SEQ ID NO: 15 - DNA, 1469 bp, Triboleum castaneum
ccatgggctataactacaccactaaatttattgatgtgccgttggaccatttctcttttaccaataacgctaccttcaagctgaaatatctgattaatgattccttttggattgatgatggtcctatcttcttttacaccggtaacgaaggtgctgtggaaactttcgcagaaaataccggttttattttcgatattgcgccaacctttaacgctctgattgttttcgccgaacacagatattacggtgcaaccctgccgtttggcaatgcgtcattctccaaccctggtcatctgggctttttgactagttctcaggctctggccgattatgtgtacctgattaatcacctgcaaaccacccatcagcgctccgaatatctgtcaaaggtcccagttgtggcattcggcggtagctacggcggcatgctggcggcttggctgcgtatgaaatatccggcctccgtggttggtgcaattgcggcttctgcccctatctggcaatttcagggtctgaccccatgtgaaaacttcaatagaattgtgtccaacgtgtacaagactgcagttgacgatgattgctcagcgccgattcaaaaatcctggaagattattcgcaatattaccgctaacgatgatggcaaagcctggctgaccaaggcatggaaactgtgttcccctctgaagtcttccgatattgatgatctgttggaatggtattcagagattctggtgaatatggcgatggtcaactacccatatccgaccaaatttctggctcctctgccagccttcccggttcgtaatttttgctacaagttgactggtgaaaaaattaccgatgataagagcctggtgaccgcaattggtaacgctctggaaatctacaccaatttcactaaagctaccaagtgtaacaatattaaccagaccgccgcaagtctgggcgaagaaggttgggattttcaagcgtgcaccgaaatgattatgcctatgtgttctgatgataatgatatgttcgaaaaccagtcctgggattttaaaaagtactcagataaatgctatactaagtggggtgtgagacaaaccaatgcagaactgccaattctggagtacggcggtaaagatattactgccgcttccaacattgttttcagtaatggtctgcttgatccttggtcttccggtggtgtgctgtcaaacatcagcagtaccgtctcttccgtgattattcctgaaggtgcccaccatctggatctgcgcggtgaaaatcgtaacgatccgaagtcagttattgaagctagacagtttcacgttagctctattcgcaaatggattactgatttctattactctcgtgataagaattattttaaaaaatccatctttttcaacaaaattacctacactcatggtaataacattctggttccacgtggaagtcatcaccatcaccatcactaaggatcc

SEQ ID NO: 16 - DNA, 1436 bp, Triboleum castaneum
ccatgggctatgattacgaaaccaaatattttgaagtgttgctggaccatttctcttttactaacaatgctaccttcaagttgaaatacctgattaacgataccttttggaccaatgatggtccgatcttcttttatactggtaacgaaggaacagtggaaaatttcgccgaaaacaccggttttatgttcgatattgcaccttcctttaatgcgttggttgtgttcgctgaacacagatactatggtgaatcactgccatttggcaacgattccttcgtgtccccgtctcatattggttacttgacctcctcacaggccctggcagattttgttgacttgattaattatctgcaaactatgagcttggagaaggtgcctgtgattgcgttcggtggaagttacggtggtatgctggcttcttggctgcgcatgaaatatccagcctccgttgtgggcgcaattgcggcttcagccccgatctggcagtttgaaaccccttgtgaagatttctacaaggtggttacgcgtgtgtatcaagaagcagtggcgaaagattgcccactgctgattaccaagtcctggactgctctgagaaacatttccgaatctccggagggtaaagcctggctgtccgatgcatggcagctgtgttcacctttggagacctccgcggatgttgaaaccctgattggttggtactccgaaattctggtgaatatggctatggtcaactatccatactctacctcctttctggccccgctgcctccattcccggttaagactttttgctcacaactgacccaggcaaatattgtggatgataaatctctggtgatggcgctgggcgatgctctgcaaatctataccaacttcaccgaaactaccacttgtaataagattaaccagaccgccgaggcactgggtgaagaaggttggtactttcaagcgtgcactgaaatgattatgcctatgtgttccattgatggcgatatgttcgaaaatgatccttgggattatggtaaatacgcttctcagtgctttgaaaagtggggtgttaaccaaacccacccggaactgcctgtgctggaatatggcggtaaagaaattaaggccgcatccaatattgtcttctcaaacggtctgcttgatccttggtcctcaggcggtgttctgaaaaatgtgtctgaatctgtggtgtccgttattattccggatggtgcgcatcacatcgatctgcgcggtggtaacaaggatgatcctgaaactgtgatcgaagcacgtcagttccatgttgataacattaaaaagtggatcatggaattttacttccactctggaaagggtgctttctttgaaaagatcaagtaccaacatgttgctagaaacctggttccacgtggaagtcatcaccatcaccatcactaaggatcc